(19) United States
(12) Patent Application Publication
(10) Pub. No.: US 2026/0174512 A1
(43) Pub. Date: Jun. 25, 2026

(54) MAGNETIC ROBOTIC ASSEMBLY, MAGNETIC ROBOTIC SYSTEM, METHOD FOR LOCATING A MEDICAL DEVICE

(71) Applicant: ATLAS ENDOSCOPY LIMITED, Leeds, Yorkshire (GB)

(72) Inventors: Bruno SCAGLIONI, Leeds (GB); Donato MAZZEO, Leeds (GB); Pietro VALDASTRI, Leeds (GB)

(21) Appl. No.: 19/128,924

(22) PCT Filed: Nov. 7, 2023
(86) PCT No.: PCT/IB2023/061235

§ 371 Date: May 9, 2025

(30) Foreign Application Priority Data
Nov. 10, 2022 (IT) 102022000023214

(51) Int. Cl. A61B 34/30; A61B 34/00; A61B 34/20

(52) U.S. Cl. CPC A61B 34/30; A61B 34/20; A61B 34/73; A61B 2034/2048; A61B 2034/2051; A61B 2034/301; A61B 2034/731

(57) ABSTRACT

A magnetic robotic assembly for magnetically locating a medical device, includes a robotic arm, and a robot base. The robotic arm is movable in multiple configurations with respect to the robot base and extends between a basal end connected to the robot base and a free end of the arm. First and second magnetic sources generate first and second magnetic fields. The first magnetic field is constant, and the second magnetic field is periodically variable. The first magnetic source is connected to the robotic arm free end and is integrally movable with the robotic arm free end in multiple positions of the first magnetic source with respect to the robot base to immerse an insertion portion in the first magnetic field. The second magnetic source is constrained to the robot base at a second magnetic source position which is fixed and integral with respect to the robot base.

### FIELD OF THE INVENTION

The present invention relates to a magnetic robotic assembly, as well as to a magnetic robotic system, as well as to a method for magnetically locating a medical device, in particular an insertion portion of a medical device.

### BACKGROUND ART

Magnetic robotic systems configured to locate a medical device immersed in a magnetic field are known in the art.

Magnetic robotic systems are known comprising a robotic arm, in which a permanent magnet is connected to a distal end of a robotic arm, so that the permanent magnet is integrally movable at the distal end of the robotic arm by immersing the medical device in the magnetic field.

The use of a magnetic robotic system provided with a single permanent magnet, due to the symmetry of the magnetic field generated by the permanent magnet, has the disadvantage of preventing the localization of the medical device in the singularities of the magnetic field, or in that plurality of points in which one or more Cartesian components of the magnetic field have the same value, preventing a unique correspondence between the value of the field detected by the medical device and the detection position.

In order to solve such a drawback, U.S. Pat. No. 11,122,965 teaches to arrange an electromagnetic coil around the permanent magnet, integrally connecting it to the permanent magnet and to the distal end of the robotic arm so as to generate a sinusoidal magnetic field with a magnetic moment perpendicular to the magnetic moment of the constant magnetic field generated by the permanent magnet. The superposition of the sinusoidal magnetic field generated by the electromagnetic coil with the constant magnetic field generated by the permanent magnet prevents the magnetic field in which the medical device is immersed from having the same value in two different points.

Although this solution allows uniquely locating the medical device immersed in the magnetic field, the arrangement of the coil wound around the permanent magnet generates intense vibrations in the permanent magnet when the coil is powered. The permanent magnet, vibrating intensely and at high frequency, on the one hand, generates strong noises which are unacceptable in robotic systems in the medical field, according to the regulations related to medical devices, and which are a danger to the health of users and operators with the robotic system, on the other hand it transmits vibrations in the robotic arm which can in turn be transmitted to the surfaces on which the robotic arm is supported, triggering stresses to the joints and electronics of the robotic system which can cause unwanted machine stops or accelerate wear phenomena.

Therefore, the need to create a magnetic robot assembly as well as a magnetic robotic system is strongly felt in the field, which allow uniquely locating medical device immersed in a magnetic field, reducing if not eliminating the noise and stresses to the robotic arm caused by the vibrations of the permanent magnet, while increasing the localization sensitivity, the positioning speed as compared to what is known.

### Solution

The present invention aims to provide a robotic assembly, as well as a robotic system, as well as a magnetic localization method, which allow generating a magnetic field and locating a medical device immersed in the uniquely generated and highly accurate magnetic field, reducing noise in the localization step.

This and other objects and advantages are achieved with a robotic assembly according to claim 1, as well as a robotic system according to claim 8, as well as a localization method according to claim 9,

Some advantageous embodiments are the subject of the dependent claims.

By virtue of the suggested solutions, it is possible to ensure an independent positioning of a first magnetic source configured to generate a permanent and/or constant magnetic field and a second magnetic source configured to generate a periodic and/or variable magnetic field, supported by the same robotic assembly, avoiding any vibration of a magnetic source caused by the other magnetic source, connecting the first magnetic source to a robotic arm movable in a plurality of configurations known and predetermined by the kinematics of the robotic arm, and connecting the second magnetic source to a robot base supporting the robotic arm, allowing the relative position between the first magnetic source and the second magnetic source to be simultaneously known for each configuration in which the robotic arm is movable.

By virtue of the suggested solutions, it is possible to ensure an independence of the dimensions of the first magnetic source from the dimensions of the second magnetic source and vice versa, allowing the creation robotic assemblies and robotic systems which can generate magnetic fields which are easily adaptable depending on the conditions in which they must operate.

By virtue of the suggested solutions, it is possible to avoid a connection with the robotic arm of the magnetic source configured to generate a periodic and/or variable magnetic field, avoiding having an electrical connection through the robotic arm to power the second magnetic source, which instead, being constrained to the robot base, can be easily powered without power constraints.

By virtue of the suggested solutions, it is possible to reduce the masses supported by the robotic arm as compared to what is known, while allowing a unique localization of the medical device immersed in the magnetic field, as well as a faster and more accurate positioning speed of the first magnetic source as compared to what is known.

### FIGURES

Further features and advantages of the magnetic robotic assembly, the magnetic robotic system, and the localization method will become apparent from the description of preferred embodiments thereof, provided by way of non-limiting indication, with reference to the accompanying drawings, in which:

FIG. 1 depicts an axonometric view of a magnetic robotic assembly according to the present invention, in which a first magnetic source can be seen, which is for generating permanent and/or constant magnetic field directly connected to the distal end of a robotic arm to be integrally movable with the robotic arm, as an effector of the magnetic robotic assembly, and a second magnetic source supported by a robot base in a fixed position with respect to any position in which said first magnetic source can be arranged, in which the second magnetic source is configured to generate a periodic and/or variable magnetic field;

FIG. 2 depicts an axonometric view of a magnetic robotic system according to the present invention, comprising the magnetic robotic assembly in FIG. 1 and medical device operatively connected to said magnetic robotic assembly, preferably an endoscope provided with an insertion portion adapted to be inserted into a cavity, and preferably connected to a flexible body connected to the robot base;

FIG. 3 depicts a partially sectioned axonometric view of the second magnetic source of the robot assembly in FIGS. 1 and 2;

FIG. 4 Shows an Axonometric View of the Insertion portion of the medical device to be located, in which a plurality of sensors supported by the insertion portion for measuring accelerations, angular velocities and magnetic field values along at least three device axes orthogonal and/or incident to each other are shown;

FIG. 5 depicts a side view of the magnetic robotic system in FIG. 2 partially sectioned, in which the lines of the permanent and/or constant magnetic field generated by the first magnetic source and the lines of the periodic and/or variable and/or periodically variable magnetic field generated by the second magnetic source are visible, in which the lines of the magnetic field generated by the first magnetic source and the lines of the magnetic field generated by the second magnetic source are incidental and/or perpendicular, avoiding any parallelism, in which the insertion portion of the medical device immersed in the lines of the magnetic field generated by the first magnetic source and in the lines of the magnetic field generated by the second magnetic source can be seen;

FIG. 6 diagrammatically depicts the localization method implemented in the robotic system in FIG. 5.

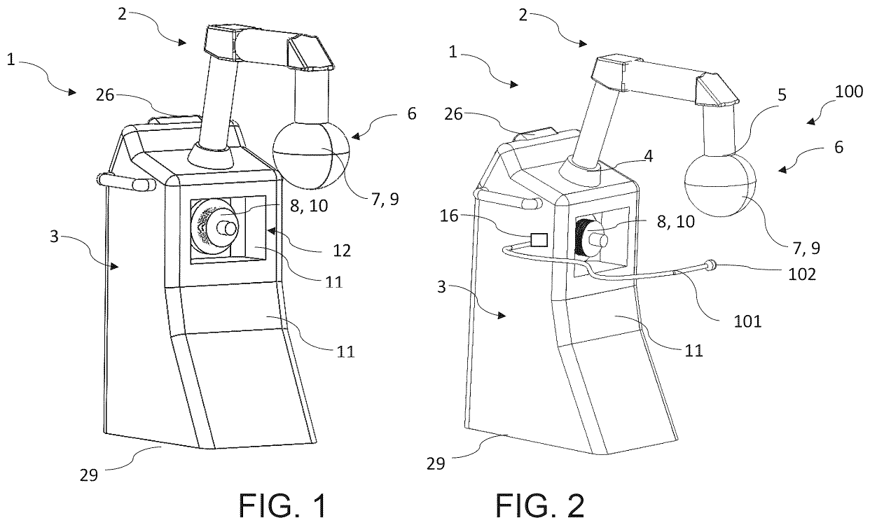

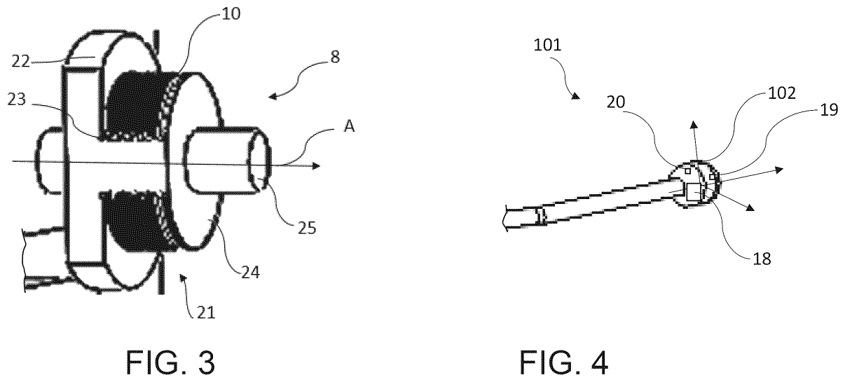

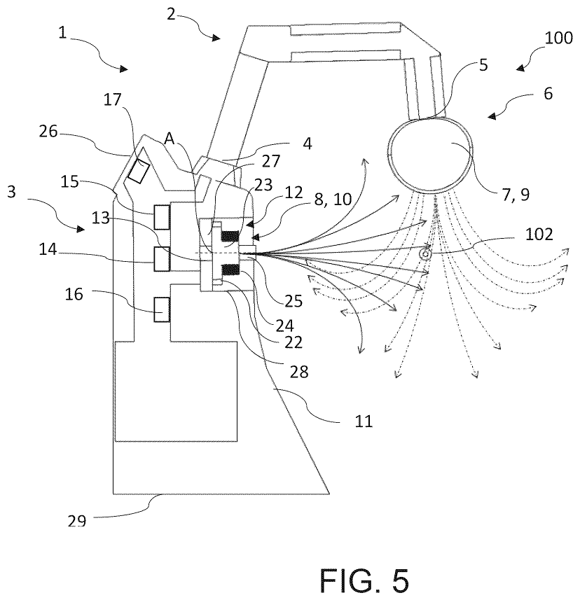

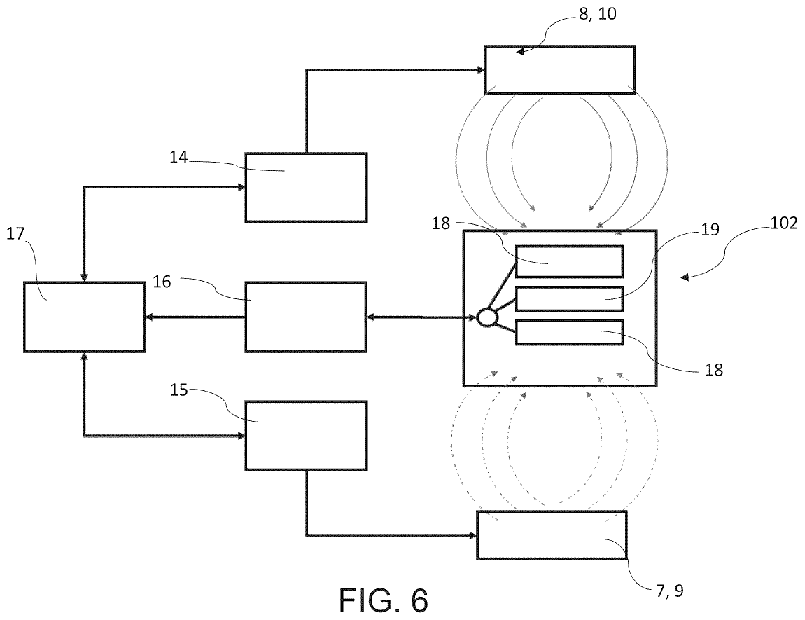

### DESCRIPTION OF SOME PREFERRED EMBODIMENTS

In accordance with a general embodiment, a magnetic robotic assembly for magnetically locating a medical device 101 is indicated by reference numeral 1 as a whole.

The magnetic robotic assembly 1 comprises a robotic arm 2 and a robot base 3 configured to support the robotic arm 2. The robotic arm 2 is movable in a plurality of configurations with respect to said robot base 3.

The robotic arm 2 extends at least between a robotic arm basal end 4 and a robotic arm distal end 5, in which said robotic arm basal end 4 is connected to said robot base 3.

The magnetic robotic assembly 1 comprises a first magnetic source 7 configured to generate a first magnetic field, in which the first magnetic field is constant.

The first magnetic source 7 is connected to said robotic arm distal end 5. The first magnetic source 7 is movable integrally with said robotic arm distal end 2 in a plurality of operating positions of the first magnetic source with respect to said robot base 3 so as to immerse at least one insertion portion 102 of the medical device 101 in the first magnetic field by moving said robotic arm 5.

The magnetic robotic assembly 1 comprises at least a second magnetic source 8 configured to generate a second magnetic field, in which the second magnetic field is periodically variable.

Advantageously, said at least a second magnetic source 8 is constrained to said robot base 3 so as to be arranged in a second magnetic source position which is fixed and integral with respect to said robot base 3 for each first magnetic source position of said first magnetic source 7.

By virtue of the positioning of the at least a second magnetic source 8 in a fixed and integral position with the robot base 3 and of the first magnetic source 7 in a position integral with the robotic arm distal end 5, it is possible to provide a magnetic robotic assembly having two magnetic sources positioned, one movable and one fixed, in positions independent of each other, in which it is possible to know a priori, the relative position between the first magnetic source 7, movable, with respect to the at least a second magnetic source 8, the kinematics with which the robotic arm 2 moves in space being known.

By virtue of the positioning of the at least a second magnetic source 8 in a fixed and integral position with the robot base 3, it is possible to avoid or at least reduce a magnetic interaction between the second magnetic field and the first magnetic source 7, avoiding causing unwanted vibrations in the first magnetic source 7 and therefore in the robotic arm 2.

In accordance with an embodiment, said at least a second magnetic source 8 is oriented so that when said first magnetic source 7 is in any first magnetic source operating position of said plurality of first magnetic source operating positions, the second magnetic field is superimposed on the first magnetic field.

In accordance with an embodiment, said at least a second magnetic source 8 is oriented so to immerse at least the insertion portion 102 in the first magnetic field and in the second magnetic field.

In accordance with an embodiment, said at least a second magnetic source 8 is oriented so as to avoid a parallelism between the field lines and/or the magnetic field vectors of said second magnetic field and the field lines and/or the magnetic field vectors of said first magnetic field in any position in which the first magnetic field is superimposed on the second magnetic field.

In accordance with an embodiment, said first magnetic source 7 is connected to said robotic arm distal end 5, avoiding electrical connections for electrically powering said first magnetic source 7 between said first magnetic source 7 and said robotic arm 2 and/or said robot base 3. High power electrical connections supported by the robotic arm 2 can thus be avoided.

In accordance with an embodiment, said magnetic robotic assembly 1 comprises an effector 6 directly connected to said robotic arm distal end 5, in which said effector 6 comprises said first magnetic source 7 avoiding supporting further magnetic sources configured to generate a respective further magnetic field.

In accordance with an embodiment, said first magnetic source 7 is a permanent magnet 9 configured to generate said first magnetic field.

In accordance with an embodiment, said at least one magnetic source 8 is an electromagnet configured to generate said second magnetic field.

In accordance with an embodiment, said at least a second magnetic source 8 comprises at least one winding 10 configured to generate said second magnetic field when crossed by a current.

In accordance with an embodiment, said robot base 3 comprises a base body 11 delimiting at least one seat 12 of second magnetic source, in which said at least a second magnetic source 8 is housed in said seat 12 of second magnetic source in connection with said robot base 3.

In accordance with an embodiment, said at least one

magnetic source 8 is electrically powered by an electrical connection of a second magnetic source, in which said electrical connection is housed inside said base body 11. It is thus possible to avoid the presence of electrical connections of second magnetic source reachable outside the robot base 3, and/or which can be hindering in a zone in which the robot assembly 1 operates and/or can form an electrical hazard for an operator of the robot assembly or for a patient on whom the robot assembly operates.

In accordance with an embodiment, said robot base 3 comprises a support portion 13 adapted to directly support said at least a second magnetic source 8. In accordance with an embodiment, said support portion 13 at least partially delimits said at least a seat 12 of second magnetic source.

In accordance with an embodiment, said robot base 3 comprises reversible connection means 13 configured to reversibly connect and/or form a reversible coupling between said at least a second magnetic source 8 and said robot base 3. In accordance with an embodiment, the reversible coupling between the robot base 3 and the second magnetic source is a reversible shape coupling between a portion of the second magnetic source and a portion of the robot base. In accordance with an embodiment, said reversible connection means 13 are coupling means configured to couple the second magnetic source to the robot base 3.

In accordance with embodiment, the reversible coupling between said at least a second magnetic source 8 and said robot base 3 allows replacing the second magnetic source 8 connected to the robot base with another second magnetic source 8. In accordance with an embodiment, a robotic assembly kit comprises a robotic assembly according to one of the embodiments described herein and a set of second magnetic sources, in which each second magnetic source is reversibly connectable to the robot base 3 and is replaceable with another second magnetic source of the set of magnetic sources.

In accordance with an embodiment, said at least a second magnetic source 8 comprises a coil support 21 adapted to support said at least one winding 10, in which said coil support 21 has at least one hollow portion to electrically connect the at least one winding 10.

In accordance with an embodiment, the coil support 21

comprises at least one bottom plate 22 connected to a winding portion 23, preferably tubular, in which said at least one winding 10 is wound around said winding portion 23. In accordance with an embodiment, said coil support 21 comprises a front plate 24 adapted to couple to the winding portion 23 on an opposite side with respect to the bottom plate 22.

In accordance with an embodiment, the at least a second magnetic source 8 comprises a magnetic core 25 so as to concentrate the lines of the second magnetic field in the material of the magnetic core 25. In accordance with an embodiment, the magnetic core 25 is coaxial with said winding portion 23 and/or with said winding. In accordance with an embodiment, the magnetic core 25 is connected to said front plate 24 at least on the opposite side to said winding portion 23.

In accordance with an embodiment, said robot base 3 comprises a control panel 26 adapted to control said magnetic robotic assembly 1. In accordance with an embodiment, said magnetic robotic assembly 1 defines a front zone in which the robotic arm distal end 5 is mainly adapted to move. In accordance with an embodiment, the control panel 26 is arranged on the base body 11 in a zone opposite to the front zone.

In accordance with an embodiment, said base body 4 avoids shielding the second magnetic field at least towards said front part of the magnetic robotic assembly 1.

In accordance with an embodiment, said base body 11 shields the second magnetic field at least towards the control panel.

In accordance with an embodiment, said base body 4 comprises at least one seat bottom wall 27 and at least one seat side wall 28 delimiting said at least one seat 12 of second magnetic source.

In accordance with an embodiment, said at least one seat side wall 28 delimits a seat opening 29 with an edge thereof. In accordance with an embodiment, said seat opening 29 is opposite to said at least one seat bottom wall 27 allowing an access to said at least one seat 12 of second magnetic source from an environment outside the base body 4, for example from said front zone.

In accordance with an embodiment, said at least one seat bottom wall 27 and said at least one seat side wall 28 are connected so as to define a box-like shape, for example cylindrical or prismatic.

In accordance with an embodiment, said at least one seat bottom wall 27 and said at least one seat side wall 28 are configured to shield the second magnetic field generated by said at least a second magnetic source 8 avoiding shielding in the direction of said seat opening 29.

In accordance with an embodiment, said at least one winding 10 defines a winding axis A. In accordance with an embodiment, said robot base 3 comprises a pedestal 29 adapted to rest on a support surface or a floor to stably support the robotic arm 2.

In accordance with an embodiment, the pedestal 29 comprises an abutment plane, in which the winding axis A is parallel to the abutment plane.

In accordance with an embodiment, the winding axis A forms with the abutment plane of said pedestal 29 a winding angle between 0 and 60 degrees, preferably between 0 and 45 degrees.

In accordance with an embodiment, said robotic arm 2 comprises at least one plurality of rigid links or connections, and a plurality of rotational joints which allow a rotation of one link with respect to another with a rotational degree of freedom. In accordance with an embodiment, said plurality of rigid links or connections comprises at least one distal link and one basal link. In accordance with an embodiment, said distal link comprises said robotic arm distal end 5. In accordance with an embodiment, said basal link comprises said robotic arm basal end 4.

The present invention further relates to a magnetic robotic system 100 for magnetically locating a medical device 101. The magnetic robotic system 100 comprises at least one magnetic robotic assembly 1 according to any of the described embodiments.

The magnetic robotic system 100 comprises at least said medical device 101, in which said medical device 101 comprises an insertion portion 102 adapted to be introduced into a cavity.

The insertion portion 102 comprises a plurality of magnetic field sensors 18 configured to detect a three-dimensional magnetic field vector and/or at least three magnetic field components with respect to at least three mutually incident axes of a magnetic field in which said insertion portion 102 is immersed. In accordance with an embodiment, the plurality of magnetic field sensors 18 is configured to detect magnetic field signals. In accordance with an embodiment, said insertion portion 102 defines at least three first device axes, in which said at least three first device axes are incidental and/or orthogonal to each other. In accordance with an embodiment, said plurality of magnetic field sensors 18 is at least three magnetic field sensors configured to each detect a respective component of the three-dimensional magnetic field vector with respect to a respective first device axis. In accordance with an embodiment, said plurality of magnetic field sensors 18 comprises at least one Hall-effect sensor.

The insertion portion 102 comprises a triaxial accelerometer 19 configured to detect a three-dimensional vector of acceleration and/or at least three accelerations with respect to three mutually incident axes of said insertion portion 102. In accordance with an embodiment, said device insertion portion 102 defines at least three second device axes, in which the at least three second device axes are mutually incidental and/or orthogonal. In accordance with an embodiment, said triaxial accelerometer 19 is configured to detect each component of said three-dimensional acceleration vector with respect to each of said at least three second device axes.

The insertion portion 102 comprises a triaxial gyroscope 20 configured to detect at least three angular velocities of said insertion portion 102 with respect to at least three mutually incident and/or orthogonal axes. In accordance with an embodiment, said device insertion portion 102 defines at least three third device axes, in which the at least three third device axes are mutually incidental and/or orthogonal. In accordance with an embodiment, said triaxial gyroscope 20 is configured to detect an angular velocity with respect to each of said at least three device axes.

The magnetic robotic system 100 comprises a first magnetic source position control unit 15 configured to detect the position of the first magnetic source between said plurality of first magnetic source positions of the first magnetic source 7.

The magnetic robotic system 100 comprises a second magnetic field control unit 14 configured to control a predefined frequency and a predefined periodic waveform to generate said second magnetic field with said at least a second magnetic source 8. In accordance with an embodiment, said predefined periodic waveform is a sine wave and/or square wave and/or triangular wave and/or sawtooth wave. In accordance with an embodiment, said predefined periodic waveform has said predefined frequency. In accordance with an embodiment, said predefined frequency is at least 200 Hz.

The magnetic robotic system 100 comprises a processing unit 17 configured to calculate and/or estimate a roll and a pitch of said insertion portion 102 with respect to a reference point of said robot base 4 based on said three-dimensional acceleration vector and said at least three angular velocities detected by said triaxial accelerometer 19 and said triaxial gyroscope 20, respectively.

Advantageously, said processing unit 17 is configured to calculate and/or estimate a medical device position Xp, Yp, Zp of said insertion portion 102 and a yaw of said insertion portion 102 with respect to said reference point of said robot base 4 based on the calculated roll, the calculated pitch, the detected three-dimensional magnetic field vector, a relative position of magnetic sources defined by the difference between the detected first magnetic source position and the second magnetic source position fixed and integral with the robot base 4, a relative position of second magnetic source defined by the difference between the second magnetic source position fixed and integral with the robot base 4 and the reference point of said robot base 4, from the predefined frequency and the predefined periodic waveform.

In accordance with an embodiment, said first magnetic source position control unit 15, said second magnetic field control unit 14, and said processing unit 17 are housed in housing obtained in said robot base 3. In accordance with an embodiment, said first magnetic source position control unit 15 is operatively connected to said robotic arm 2. In accordance with an embodiment, the second magnetic field control unit 14 is operatively connected to said at least a second magnetic source 8.

In accordance with an embodiment, said medical device 101 is an endoscope directly connected to said robotic assembly 1, and in which said insertion portion 102 is the tip of the endoscope, in which said insertion portion 102 is connected to the robotic assembly 1 by means of a flexible endoscope body or shaft.

In accordance with an embodiment, the magnetic robotic system 100 comprises a data transceiver unit 16 configured to receive at a detection frequency said three-dimensional magnetic field vector, said three-dimensional acceleration vector and said at least three angular velocities, from said plurality of magnetic field sensors 18, from said triaxial accelerometer 19 and from said triaxial gyroscope 20, respectively; in which said data transceiver unit 16 is configured to transmit said detected three-dimensional magnetic field vector, said detected three-dimensional acceleration vector and said at least three angular velocities, to said processing unit 17.

In accordance with an embodiment, said processing unit 17 comprises a memory unit for storing one or more of: said detected three-dimensional magnetic field vector, said detected three-Dimensional acceleration vector, said at least three detected angular velocities, said calculated roll, said calculated pitch, said relative position of magnetic sources, said relative position of second magnetic source, said reference point of said robot base 4, said predefined frequency and said predefined waveform.

In accordance with an embodiment, said processing unit 17 is operatively connected to said effector position control unit 15 for acquiring, storing, and calculating an average of the relative position between said first magnetic source 7 and said at least a second magnetic source 8.

In accordance with an embodiment, said processing unit 17 is operatively connected to said data transceiver unit 16 for acquiring, storing, and calculating a first function of the detected three-dimensional acceleration vectors, a second function Of the detected three-dimensional angular velocity vectors, and an average Of the detected three-dimensional magnetic field vectors. In accordance with an embodiment, said first function and/or said second function are a mathematical function such as a mean, a median or a function referring to the last detected sample.

In accordance with an embodiment, said processing unit 17 is operatively connected to said second magnetic field control unit 16 for acquiring and storing the waveform of the second magnetic field, and for calculating amplitude and phase of the detected magnetic field.

The present invention further relates to a method for locating a medical device 101 immersed in a magnetic field. The medical device 101 comprises an insertion portion 102 adapted to be introduced into a Said insertion portion 102 comprises a plurality of magnetic field sensors 18 configured to detect a three-dimensional magnetic field vector of said magnetic field in which said insertion portion 102 is immersed. Said insertion portion 102 comprises a triaxial accelerometer 19 configured to detect a three-dimensional vector of acceleration of said insertion portion 102. Said insertion portion 102 comprises a triaxial gyroscope 20 configured to detect at least three angular velocities of said insertion portion 102 with respect to at least three mutually incident axes.

The method comprises the steps of:
providing a robot assembly 1 according to any of the described embodiments;
generating, by said first magnetic source 7 and said second magnetic source 8, said first magnetic field and said second magnetic field, respectively, that the medical device 101 is immersed at least in the superposition of the first magnetic field and the second magnetic field,
keeping the first magnetic field constant,
periodically varying the second magnetic field with a predefined periodic wave function and a predefined frequency,
detecting the position of the second magnetic source fixed and integral with the robot base 4,
defining a reference position of said robot base 4,
calculating a relative position of second magnetic source defined by the difference between the second magnetic source position and the reference position,
detecting, acquiring and storing the first magnetic source position of the first magnetic source 7,
calculating relative position of magnetic sources defined by the difference between the detected first magnetic source position and the second magnetic source position fixed and integral with the robot base 4,
detecting, acquiring and storing at an acquisition frequency said three-dimensional magnetic field vector, said three-dimensional acceleration vector and said at least three angular velocities,
calculating and storing a roll and a pitch of said insertion portion 102 with respect to a reference point of said robot base 4 based on said three-dimensional acceleration vector and said at least three angular velocities detected by said triaxial accelerometer 19 and by said triaxial gyroscope 20, respectively,
calculating a medical device position Xp, Yp, Zp of said insertion portion 102 and a yaw of said insertion portion 102 with respect to said reference point of said robot base 4 based on the calculated roll, the calculated pitch, the calculated relative position of magnetic sources, the calculated relative position of second magnetic source, the three-dimensional magnetic field vector, the predefined periodic wave function, and the predefined frequency.

In accordance with an operating mode, the method comprises the step of providing a robot system 100 according to any one of the described embodiments.

In accordance with an operating mode, the method comprises the step of—calculating and storing at a calculation frequency a first function, for example an average or median or last value detected, of the three-dimensional acceleration vector detected and/or the Cartesian components thereof detected at said acquisition frequency, a second function, for example an average or median or last value detected, of said at least three angular velocities detected at said acquisition frequency, and an average of the three-dimensional magnetic field vector detected and/or the Cartesian components thereof detected at said acquisition frequency, in which the average of the three-dimensional magnetic field vector detected at said acquisition frequency is significant of the first magnetic field detected.

In accordance with an operating mode, the method comprises the step of calculating and storing at said calculation frequency an average of the first magnetic source position of the first magnetic source 7 as an average between the first magnetic source positions detected.

In accordance with an operating mode, the method comprises the step of calculating and storing at said calculation frequency the phase and amplitude of the detected magnetic field associated with the second magnetic field source 8 based on a sequence of three-dimensional magnetic field vectors detected by the plurality of magnetic field sensors 18 in a time interval between an amplitude and phase calculation and the next, in which the magnetic field phase and amplitude calculated at the calculation frequency are significant of the second magnetic field.

In accordance with an operating mode, said roll and said pitch of said insertion portion 102 are estimated based on the first function of the three-dimensional acceleration vector, and the second function of the at least three angular velocities detected.

In accordance with an operating mode, said medical device position Xp, Yp, Zp and said yaw are estimated based on said roll calculated at said calculation frequency, said pitch calculated at said calculation frequency, said average magnetic field calculated at said calculation frequency, the magnetic field amplitude and phase calculated, the average of the first magnetic source position, on models of magnetic field generated by said first magnetic source 7 and said second magnetic source 8.

In accordance with an operating mode, said acquisition frequency is at least one order of magnitude, preferably two orders of magnitude, higher than said calculation frequency. In accordance with an operating mode, said acquisition frequency is at least 20 KHz, and said calculation frequency is at least 100 Hz.

In accordance with an operating mode, the phase and amplitude of the magnetic field detected are calculated by variants of the fast Fourier transforms, such as the Goertzel algorithm, the predefined frequency and the predefined periodic wave function being known.

In accordance with an operating mode, said roll and said pitch of said insertion portion 102 are estimated through finite and infinite response filters, such as a Mahony filter.

In accordance with an operating mode, said magnetic field models generated by said first magnetic source 7 and said second magnetic source 8 used for the estimation of said medical device position Xp, Yp, Zp and said yaw are a magnetic dipole model and/or generalized elliptical integral models.

In accordance with an operating mode, for the estimation of said medical device position Xp, Yp, Zp and said yaw, statistical observatories are used, in which said statistical observatories comprise pseudo-Montecarlo-type algorithms, such as particle filter, or other statistical filters, such as a Kalmann filter.

### LIST OF REFERENCE SIGNS

1 robotic magnetic assembly
2 robotic arm
3 robot base
4 robotic arm basal end
5 robotic arm distal end
6 effector
7 first magnetic source
8 second magnetic source
9 permanent magnet
10 winding
11 base body
12 seat of second magnetic source
13 support portion
14 second magnetic field control unit
15 first magnetic source position control unit
16 data transceiver unit
17 processing unit
18 plurality of magnetic field sensors or Hall-effect sensors
19 triaxial accelerometer
20 triaxial gyroscope
21 coil support
22 bottom plate
23 winding portion
24 front plate
25 magnetic core
26 control panel
27 seat bottom wall
28 seat side wall
29 pedestal
100 robotic magnetic system
101 medical device
102 insertion portion
A winding axis

1. A magnetic robotic assembly for magnetically locating a medical device comprising:
a robotic arm,
a robot base configured to support the robotic arm wherein the robotic arm is movable in a plurality of configurations with respect to said robot base,
wherein the robotic arm extends at least between a robotic arm basal end and a robotic arm distal end wherein said robotic arm basal end is connected to said robot base;
a first magnetic source configured to generate a first magnetic field and at least a second magnetic source configured to generate a second magnetic field, wherein the first magnetic field is constant and wherein the second magnetic field is periodically variable,
wherein the first magnetic source is connected to said robotic arm distal end wherein the first magnetic source is movable integrally with said robotic arm distal end in a plurality of operating positions of the first magnetic source with respect to said robot base to immerse at least one insertion portion of the medical device in the first magnetic field by moving said robotic arm;
wherein
said at least a second magnetic source is constrained to said robot base to be arranged in a second magnetic source position which is fixed and integral with respect to said robot base for each first magnetic source position of said first magnetic source.

2. A magnetic robotic assembly according to claim 1,
wherein said at least a second magnetic source is oriented so that when said first magnetic source is in any first magnetic source operating position of said plurality of first magnetic source operating positions, the second magnetic field is superimposed on the first magnetic field so as to immerse at least the insertion portion in the first magnetic field and the second magnetic field, and/or to avoid a parallelism between the magnetic field vectors of said second magnetic field and the magnetic field vectors of said first magnetic field in any position in which the first magnetic field is superimposed on the second magnetic field.

3. A magnetic robotic assembly according to claim 1, comprising one or more of the following features or a combination thereof:
wherein said magnetic robotic assembly comprises an effector directly connected to said robotic arm distal end wherein said effector comprises said first magnetic source avoiding supporting further magnetic sources configured to generate a respective further magnetic field; and/or
wherein said first magnetic source is a permanent magnet configured to generate said first magnetic field.

4. A magnetic robotic assembly according to claim 1, comprising one or more of the following features or a combination thereof:
wherein said at least a second magnetic source comprises at least one winding configured to generate said second magnetic field when crossed by a current, and/or
wherein said at least a second magnetic source is an electromagnet configured to generate said second magnetic field, and/or
wherein said robot base comprises a base body delimiting at least one seat of said at least a second magnetic source, wherein said at least a second magnetic source is housed in said seat of said at least a second magnetic source in connection with said robot base, and wherein said at least a second magnetic source is electrically powered by an electrical connection of a second magnetic source, wherein said electrical connection is housed inside said base body.

5. A magnetic robotic assembly according to claim 4, comprising one or more of the following features or a combination thereof:
wherein said robot base comprises a support portion adapted to directly support said at least a second magnetic source wherein said support portion at least partially delimits said at least one seat of second magnetic source; and/or
wherein said robot base comprises reversible connection means configured to reversibly connect said at least a second magnetic source to said robot base allowing a replacement of said at least a second magnetic source with another from a set of second magnetic sources; and/or
wherein said at least a second magnetic source comprises a coil support adapted to support said at least one winding wherein said coil support has at least one hollow portion to electrically connect the at least one winding, and/or
wherein the at least a second magnetic source comprises a magnetic core to concentrate the lines of the second magnetic field in the material of the magnetic core.

6. A magnetic robotic assembly according to claim 4, wherein said robot base comprises a control panel adapted to control said magnetic robotic assembly,
wherein said magnetic robotic assembly defines a front zone in which the robotic arm distal end is adapted to move, wherein the control panel is arranged on the base body in a zone opposite to the front zone,
wherein said base body avoids shielding the second magnetic field at least towards said front part of the magnetic robotic assembly, and
wherein said base body shields the second magnetic field at least towards the control panel.

7. A magnetic robotic assembly according to claim 4, wherein said at least one winding defines a winding axis, and wherein said robot base comprises a pedestal adapted to rest on a support surface or a floor to stably support the robotic arm, wherein the pedestal comprises an abutment surface, wherein the winding axis is parallel to the abutment surface or wherein the winding axis forms a winding angle between 0 and 60 degrees with the abutment surface of said pedestal.

8. A magnetic robotic system for magnetically locating a medical device, comprising:
a magnetic robotic assembly according to claim 1;
said medical device wherein said medical device comprises an insertion portion adapted to be introduced into a cavity,
wherein said insertion portion comprises a plurality of magnetic field sensors configured to detect a three-dimensional magnetic field vector of a magnetic field in which said insertion portion is immersed,
wherein said insertion portion comprises a triaxial accelerometer configured to detect a three-dimensional vector of three-dimensional acceleration of said insertion portion
wherein said insertion portion comprises a triaxial gyroscope configured to detect at least three angular velocities of said insertion portion with respect to three mutually incident and/or orthogonal axes;
a first magnetic source position control unit configured to detect the position of the first magnetic source between said plurality of first magnetic source positions of the first magnetic source,
second magnetic field control unit configured to control a predefined frequency and a predefined waveform to generate said second magnetic field with said at least a second magnetic source;
a processing unit configured to calculate and/or estimate a roll and a pitch of said insertion portion with respect to a reference point of said robot base based on said three-dimensional acceleration vector and said at least three angular velocities detected by said triaxial accelerometer and said triaxial gyroscope respectively,
wherein said processing unit is configured to calculate and/or estimate a medical device position of said insertion portion and a yaw of said insertion portion with respect to said reference point of said robot base based on the calculated roll, the calculated pitch, the detected three-dimensional magnetic field vector, a relative position of magnetic sources defined by a difference between the detected first magnetic source position and the second magnetic source position fixed and integral with the robot base, a relative position of second magnetic source defined by a difference between the second magnetic source position fixed and integral with the robot base and the reference point of said robot base from the predefined frequency and the predefined waveform.

9. A method for locating a medical device immersed in a magnetic field, wherein said medical device comprises an insertion portion adapted to be introduced into a cavity,
wherein said insertion portion comprises a plurality of magnetic field sensors configured to detect said magnetic field in which said insertion portion is immersed,
a triaxial accelerometer configured to detect a three-dimensional acceleration vector of said insertion portion,
a triaxial gyroscope configured to detect at least three angular velocities of said insertion portion with respect to three mutually incident and/or orthogonal axes;
wherein said method comprises the steps of:
providing a robot assembly according to claim 1,
generating, by said first magnetic source and said second magnetic source, said first magnetic field and said second magnetic field, respectively, so that the medical device is immersed at least in a superposition of the first magnetic field and the second magnetic field,
keeping the first magnetic field constant,
periodically varying the second magnetic field with a predefined periodic wave function and a predefined frequency,
detecting the position of the second magnetic source fixed and integral with the robot base,
defining a reference position of said robot base,
calculating a relative position of second magnetic source defined by a difference between the second magnetic source position and the reference position,
detecting, acquiring and storing the first magnetic source position of the first magnetic source,
calculating a relative position of magnetic sources defined by a difference between the detected first magnetic source position and the second magnetic source position fixed and integral with the robot base,
detecting, acquiring and storing at an acquisition frequency said three-dimensional magnetic field vector, said three-dimensional acceleration vector and said at least three angular velocities,
calculating and storing a roll and a pitch of said insertion portion with respect to a reference point of said robot base based on said three-dimensional acceleration vector and said at least three angular velocities detected by said triaxial accelerometer and said triaxial gyroscope respectively,
calculating a medical device position of said insertion portion and a yaw of said insertion portion with respect to said reference point of said robot base based on the calculated roll, the calculated pitch, the calculated relative position of magnetic sources, the calculated relative position of second magnetic source, the three-dimensional magnetic field vector, the predefined periodic wave function, and the predefined frequency.

10. A method according to claim 9 comprising the further steps of:
calculating and storing at a calculation frequency a first function, of the three-dimensional acceleration vectors detected and/or the Cartesian components thereof detected at said acquisition frequency, a second function, of the three-dimensional angular velocity vectors detected and/or the Cartesian components thereof detected at said acquisition frequency, and an average of the three-dimensional magnetic field vectors detected and/or the Cartesian components thereof detected at said acquisition frequency, wherein the average of the three-dimensional magnetic field vector detected at said acquisition frequency is significant of the first magnetic field detected,
calculating and storing at said calculation frequency an average of the first magnetic source position of the first magnetic source as an average between the first magnetic source positions detected,
calculating and storing at said calculation frequency a phase and amplitude of the detected magnetic field associated with the second magnetic field source based on a sequence of three-dimensional magnetic field vectors detected by the plurality of magnetic field sensors in a time interval between an amplitude and phase calculation and a next phase, wherein the magnetic field phase and amplitude calculated at the calculation frequency are significant of the second magnetic field,
wherein said roll and a pitch of said insertion portion are estimated based on the first function of the three-dimensional acceleration vector and the second function of said at least three angular speeds detected;
wherein said medical device position and said yaw are estimated based on said roll calculated at said calculation frequency, said pitch calculated at said calculation frequency, said average magnetic field calculated at said calculation frequency, the magnetic field amplitude and phase calculated, the average of the first magnetic source position, on models of magnetic field generated by said first magnetic source and said second magnetic source.

11. A method according to claim 10,
wherein said acquisition frequency is at least one order of magnitude, higher than said calculation frequency.

12. A method according to claim 10, wherein said acquisition frequency is at least two orders of magnitude higher than said calculation frequency.

13. A method according to claim 9, comprising the further steps of:
calculating and storing at a calculation frequency a first function comprising an average or median or last value detected, of the three-dimensional acceleration vectors detected and/or the Cartesian components thereof detected at said acquisition frequency, a second function comprising an average or median or last value detected, of the three-dimensional angular velocity vectors detected and/or the Cartesian components thereof detected at said acquisition frequency, and an average of the three-dimensional magnetic field vectors detected and/or the Cartesian components thereof detected at said acquisition frequency, wherein the average of the three-dimensional magnetic field vector detected at said acquisition frequency is significant of the first magnetic field detected,
calculating and storing at said calculation frequency an average of the first magnetic source position of the first magnetic source as an average between the first magnetic source positions detected,
calculating and storing at said calculation frequency a phase and amplitude of the detected magnetic field associated with the second magnetic field source based on a sequence of three-dimensional magnetic field vectors detected by the plurality of magnetic field sensors in a time interval between an amplitude and phase calculation and a next phase, wherein the magnetic field phase and amplitude calculated at the calculation frequency are significant of the second magnetic field,
wherein said roll and a pitch of said insertion portion are estimated based on the first function of the three-dimensional acceleration vector and the second function of said at least three angular speeds detected;
wherein said medical device position and said yaw are estimated based on said roll calculated at said calculation frequency, said pitch calculated at said calculation frequency, said average magnetic field calculated at said calculation frequency, the magnetic field amplitude and phase calculated, the average of the first magnetic source position, on models of magnetic field generated by said first magnetic source and said second magnetic source.
